(19) United States
(12) Patent Application Publication
(10) Pub. No.: US 2026/0021293 A1
(43) Pub. Date: Jan. 22, 2026

(54) MEDICAL DEVICE FIXATION WITH ANTI-ROTATION FEATURE

(71) Applicant: Medtronic, Inc., Minneapolis, MN (US)

(72) Inventors: Thomas A. Anderson, New Hope, MN (US); Zhongping C. Yang, Woodbury, MN (US)

(21) Appl. No.: 18/998,014

(22) PCT Filed: Jul. 12, 2023
(86) PCT No.: PCT/IB2023/057165

§ 371 Date: Jan. 23, 2025

Related U.S. Application Data
(60) Provisional application No. 63/369,456, filed on Jul. 26, 2022.

(51) Int. Cl. A61N 1/05

(52) U.S. Cl. CPC A61N 1/0573

(57) ABSTRACT

A fixation device comprising a first elongated body extending distally from a distal end of an implantable medical device and a second elongated body extending distally from the distal end of the implantable medical device. The first elongated body comprises a helix having one or more coils, wherein a distal end of the helix is configured to penetrate into tissue of a patient. The second elongated body is configured to flexibly maintain contact with the tissue without penetrating the tissue, and wherein the second elongated body is disposed on the distal end at a separation angle away from an electrode disposed on the distal end of the implantable medical device, wherein the separation angle comprises an angle between the electrode and the second end of the second elongated body.

This application claims the benefit of U.S. Provisional Patent Application Ser. No. 63/369,456, filed 26 Jul. 2022, the entire content of which is incorporated herein by reference.

### TECHNICAL FIELD

The disclosure relates to medical devices, and more particularly to fixation of medical devices.

### BACKGROUND

Various types of implantable medical devices (IMDs) have been implanted for treating or monitoring one or more conditions of a patient. Such IMDs may be adapted to monitor or treat conditions or functions relating to heart, muscle, nerve, brain, stomach, endocrine organs or other organs and their related functions. Such IMDs may be associated with leads that position electrodes at a desired location, or may be leadless with electrodes integrated with and/or attached to the device housing. These IMDs may have the ability to wirelessly transmit data either to another device implanted in the patient or to another instrument located externally of the patient, or both.

A cardiac pacemaker is an IMD configured to deliver cardiac pacing therapy to restore a more normal heart rhythm. Such IMDs sense the electrical activity of the heart, and deliver cardiac pacing based on the sensed electrical activity, via electrodes. Some cardiac pacemakers are implanted a distance from the heart and coupled to one or more leads that intravascularly extend into the heart to position electrodes with respect to cardiac tissue. Some cardiac pacemakers are sized to be completely implanted within one of the chambers of the heart and may include electrodes integrated with or attached to the device housing rather than leads. Some cardiac pacemakers provide dual chamber functionality, by sensing and/or stimulating the activity of both atria and ventricles, or other multi-chamber functionality. A cardiac pacemaker may provide multi-chamber functionality via leads that extend to respective heart chambers, or multiple cardiac pacemakers may provide multi-chamber functionality by being implanted in respective chambers.

### SUMMARY

In general, this disclosure is directed to configurations of fixation devices of implantable medical devices. More particularly, this disclosure is directed to implantable medical devices having fixation devices and having one or more anti-rotation features that may resist rotation and/or dislodgement of the device, e.g., due to movement of heart tissue into which the device has been fixated. In some examples, the fixation device includes a first elongated body extending from a distal end of the device and configured to penetrate tissue of a patient. The first elongated body may define a helix. In some examples, the fixation device is embodied as one or more electrodes of the implantable medical device. In some examples, the anti-rotation feature includes a second elongated body extending distally from a distal end of the device. The second elongated body may have a free end and may be positioned at a user-determined angle away from one or more electrodes of the implantable medical device. The second elongated body may resist counter-rotation of the helix out of the tissue, e.g., by compressing the tissue.

In some examples, the anti-rotation feature may improve conductivity between tissue and one or more electrodes of the implantable medical device. The anti-rotation feature (e.g., the second elongated body) may reduce the likelihood and/or frequency of a loss of electrical connection between the tissue and the one or more electrodes and facilitate more consistent delivery of electrical signals from the one or more electrodes to the tissue.

In some examples, a single implantable medical device implanted in one chamber that is able to sense in and/or deliver cardiac pacing to more than one chamber, which may avoid the need for a leaded device or multiple smaller devices to provide such functionality, which may reduce the amount of material implanted within the patient. In some examples, such an implantable medical device includes a first electrode that is configured to penetrate through wall tissue of the heart chamber in which the device is implanted, and into wall tissue of another heart chamber. In addition to the first electrode, the device includes a second electrode configured to contact the wall tissue of the chamber in which the device is implanted, e.g., without penetration of the wall tissue. The electrodes can be connected to a distal end of the device. The first electrode may be a helix configured to penetrate tissue of the patient. The implantable medical device may further include one or more anti-rotation features (e.g., the second elongated body) that prevents rotation of the device. In addition to avoiding dislodgment of the device, anti-rotation features in such implantable medical devices may prevent or reduce counter-rotation that may cause the first electrode and second electrode to lose contact with their respective intended cardiac tissue. In some examples, anti-rotation features in such implantable medical devices may reduce oscillation of the device and increase the reliability of electrical contact between second electrode and the wall tissue.

In one example, this disclosure is directed to a fixation device comprising: a first elongated body extending distally from a distal end of an implantable medical device, the first elongated body comprising: a helix having one or more coils, wherein a distal end of the helix is configured to penetrate into tissue of a patient; and a second elongated body extending distally from the distal end of the implantable medical device, wherein the second elongated body is separate from the first elongated body, wherein the second elongated body is connected to the implantable medical device at a first location and when unstressed is furthest distally from the implantable medical device at a second location, wherein the second elongated body is configured to flexibly maintain contact with the tissue without penetrating the tissue, and wherein the second elongated body is disposed on the distal end of the implantable medical device at a separation angle away from an electrode disposed on the distal end of the implantable medical device, wherein the separation angle comprises an angle between the electrode and the second location of the second elongated body.

In another example, this disclosure is directed to a device comprising: an elongated housing that extends from a proximal end to a distal end, the elongated housing configured to be implanted wholly within a first chamber of a heart, the first chamber of the heart having wall tissue; a first electrode extending distally from the distal end of the elongated housing, the first electrode comprising: a first elongated body defining a helix, wherein the helix is configured to penetrate into wall tissue of a second chamber of the heart that is separate from the first chamber of the heart; a second electrode disposed on the distal end of the elongated housing; a second elongated body extending distally from the distal end of the elongated housing, wherein the second elongated body is connected to the distal end at a first location and when unstressed is furthest distally from the distal end of the elongated housing at a second location, wherein the second elongated body is separate from the first electrode and the second electrode, wherein the second elongated body is configured to maintain contact between the second electrode and the wall tissue of the first chamber without penetrate of the wall tissue of the first chamber by the second elongated body, and wherein the second elongated body is disposed on the distal end at a separation angle away from an electrode disposed on the distal end of the implantable medical device, wherein the separation angle comprises an angle between the electrode and the second location of the second elongated body; and signal generation circuitry within the elongated housing, the signal generation circuitry being coupled to the first electrode and the second electrode, wherein the signal generation circuitry is configured to deliver cardiac pacing to the second chamber via the first electrode and the first chamber via the second electrode.

In another example, this disclosure is directed to a method comprising: delivering cardiac pacing from a device to a heart, wherein the device comprises: an elongated housing extending from a proximal end to a distal end, the elongated housing configured to be implanted wholly within a first chamber of the heart, the first chamber of the heart having wall tissue; a first electrode extending distally from the distal end of the elongated housing, the first electrode comprising: a first elongated body defining a helix; a second electrode disposed on the distal end of the elongated housing; a second elongated body extending from the distal end of the elongated housing, wherein the second elongated body is connected to the distal end at a first location and when unstressed is furthest distally from the distal end of the elongated housing at a second location, wherein the second elongated body is separate from the first electrode and the second electrode, and wherein the second elongated body is configured to flexibly maintain contact between the second electrode and the wall tissue of the first chamber without penetration of the wall tissue of the first chamber by the second electrode or the second elongated body. wherein the second elongated body is disposed on the distal end at a separation angle away from an electrode disposed on the distal end of the implantable medical device, wherein the separation angle comprises an angle between the electrode and the second location of the second elongated body; and signal generation circuitry within the elongated housing, the signal generation circuitry being coupled to the first electrode and the second electrode, wherein delivering the cardiac pacing comprises: delivering cardiac pacing to a second chamber via the first electrode; and delivering cardiac pacing to the first chamber via the second electrode.

In another example, this disclosure is directed to a fixation device comprising: a first elongated body extending distally from a distal end of an implantable medical device, the first elongated body comprising: a helix having one or more coils, wherein a distal end of the helix is configured to penetrate into tissue of a patient; and a second elongated body extending distally from the distal end of the implantable medical device, wherein the second elongated body is separate from the first elongated body, wherein the second elongated body is configured to exert a proximally-directed force on a portion of the distal end of the implantable medical device, and wherein the second elongated body is disposed on the distal end of the implantable medical device away from an electrode disposed on the distal end of the implantable medical device, such that action of the second elongated body tends to urge the electrode towards contact with the tissue.

This summary is intended to provide an overview of the subject matter described in this disclosure. It is not intended to provide an exclusive or exhaustive explanation of the methods and systems described in detail within the accompanying drawings and description below.

### BRIEF DESCRIPTION OF DRAWINGS

The details of one or more examples of this disclosure are set forth in the accompanying drawings and the description below. Other features, objects, and advantages of this disclosure will be apparent from the description and drawings, and from the claims.

FIG. 1 is a conceptual diagram illustrating an example device implanted in the heart of a patient, in accordance with one or more aspects of this disclosure.

FIG. 2 is a perspective diagram illustrating the example device of FIG. 1, in accordance with one or more aspects of this disclosure.

FIG. 3 is a functional block diagram illustrating an example configuration of the IMD of FIGS. 1 and 2, in accordance with one or more aspects of this disclosure.

FIG. 4 is a conceptual diagram of the device of FIGS. 1-3 implanted at a target implant site.

FIGS. 5A, 5B, and 5C are partial views of the device of FIGS. 1-4 from different perspectives, in accordance with one or more aspects of this disclosure.

FIG. 6A is a conceptual diagram illustrating a partial view of the device of FIGS. 1-5C.

FIG. 6B is a conceptual diagram illustrating another partial view of the device of FIGS. 1-5C.

FIG. 7 is a flow diagram illustrating an example process for deploying an example device.

### DETAILED DESCRIPTION

In general, this disclosure is directed to configurations of fixation devices of implantable medical devices. More particularly, this disclosure is directed to implantable medical devices having fixation devices and one or more anti-rotation features that may resist rotation and/or dislodgement of the device, e.g., due to movement of heart tissue into which the device has been fixated. The anti-rotation features may resist counter-rotation of the fixation devices out of the tissue.

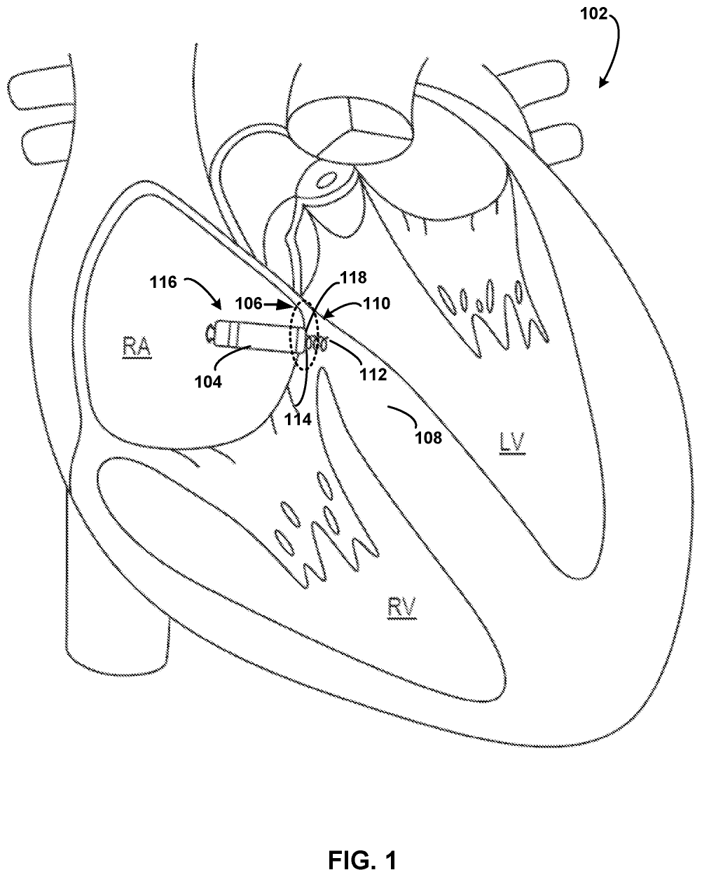

FIG. 1 is a conceptual diagram illustrating an example device 104 implanted in the heart 102 of a patient, in accordance with one or more aspects of this disclosure. Device 104 is shown implanted in the right atrium (RA) of the patient's heart 102 in a target implant region 106, such as the triangle of Koch, in heart 102 of the patient with a distal end of device 104 directed toward the left ventricle (LV) of the patient's heart 102. Although in the example of FIG. 1 the distal end of device 104 is directed toward the LV, the distal end may be directed to other targets, such as interventricular septum of heart 102. Target implant region 106 may lie between the bundle of His and the coronary sinus and may be adjacent the tricuspid valve.

Device 104 includes a distal end 110 and a proximal end 116. Distal end 110 includes a first electrode 112, a second electrode 118, and elongated body 114. First electrode 112 may define a helical shape, e.g., as illustrated in FIG. 1. First electrode 112 extends from distal end 110 and may penetrate through the wall tissue of a first chamber (e.g., the RA in the illustrated example) into wall tissue of a second chamber (e.g., ventricular myocardium 108 of the LV in the illustrated example). Elongated body 114 extends from distal end 110 and is configured to flexibly maintain contact with the wall tissue of the first chamber without penetration of the wall tissue of the first chamber by the elongated body 114 or second electrode 118. Second electrode 118 may contact the wall tissue of the first chamber as first electrode 112 penetrates the wall tissue of the first chamber and elongated body 114 flexibly maintains contact with the wall tissue of the first chamber.

The configuration of electrodes 112 and 118 illustrated in FIG. 1 allows device 104 to sense cardiac signals and/or deliver cardiac pacing to multiple chambers of heart 102, e.g., the RA and ventricle(s) in the illustrated example. In this manner, the configuration of electrodes 112 and 118 may facilitate the delivery of A-V synchronous pacing by single device 104 implanted within the single chamber, e.g., the RA. While device 104 is implanted at target implant region 106 to sense in and/or pace the RA and ventricle(s) in the example shown in FIG. 1, a device having an electrode configuration in accordance with the examples of this disclosure may be implanted at any of a variety of locations to sense in and/or pace any one, two or more chambers of heart 102. For example, device 104 may be implanted at region 106 or another region, and first electrode 112 may extend into tissue, e.g., myocardial tissue, of the LV or interventricular septum to, for example, facilitate the delivery of A-V synchronous pacing. Furthermore, a device having an electrode configuration in accordance with the examples of this disclosure may be implanted at any of a variety of locations within a patient for sensing and/or delivery of therapy to other patient tissue.

Additionally, the anti-rotation features described herein are described primarily in the context of a cardiac pacemaker configured to be implanted in one chamber and deliver pacing and sense in that chamber and an additional chamber. However, the anti-rotation features described herein may be included on any implantable medical device, such as an implantable stimulator or implantable lead configured to be fixed at any location or tissue of the body.

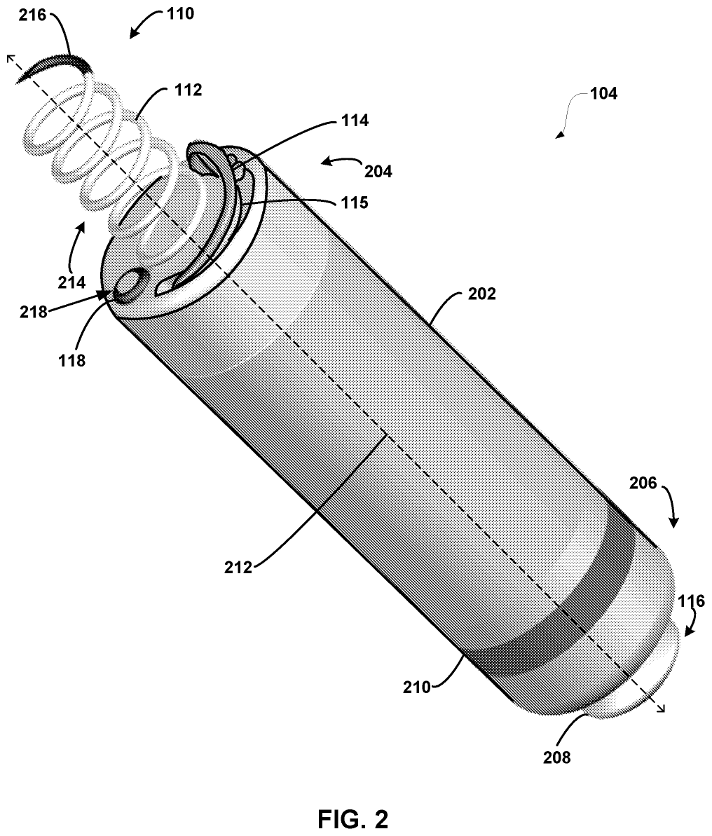

FIG. 2 is a perspective diagram illustrating device 104. Device 104 includes a housing 202 that defines a hermetically sealed internal cavity. Housing 202 may be formed from a conductive material including titanium or titanium alloy, stainless steel, MP35N (a non-magnetic nickel-cobalt-chromium-molybdenum alloy), platinum alloy or other bio-compatible metal or metal alloy, or other suitable conductive material. In some examples, housing 202 is formed from a non-conductive material including ceramic, glass, sapphire, silicone, polyurethane, epoxy, acetyl co-polymer plastics, polyether ether ketone (PEEK), a liquid crystal polymer, other biocompatible polymer, or other suitable non-conductive material.

Housing 202 extends between distal end 204 and proximal end 206. In some examples, housing can be cylindrical or substantially cylindrical but may be other shapes, e.g., prismatic, or other geometric shapes. Housing 202 may include a delivery tool interface member 208, e.g., at proximal end 206, for engaging with a delivery tool during implantation of device 104. At distal end 204, housing 202 may define a face of housing 202. The face of housing 202 may be orthogonal to longitudinal axis 212.

All, substantially all, or a portion of housing 202 may function as an electrode 210, e.g., an anode, during pacing and/or sensing. In some examples, electrode 210 can circumscribe a portion of housing 202 at or near proximal end 206. Electrode 210 can fully or partially circumscribe housing 202. FIG. 2 shows electrode 210 extending as a singular band. Electrode 210 can also include multiple segments spaced a distance apart along a longitudinal axis 212 of housing 202 and/or around a perimeter of housing 202.

When housing 202 is formed from a conductive material, such as a titanium alloy, portions of housing 202 may be electrically insulated by a non-conductive material, such as a coating of parylene, polyurethane, silicone, epoxy or other biocompatible polymer, or other suitable material. For the portions of housing 202 without the non-conductive material, one or more discrete areas of housing 202 with conductive material can be exposed to define electrode 210.

When housing 202 is formed from a non-conductive material, such as a ceramic, glass or polymer material, an electrically-conductive coating or layer, such as a titanium, platinum, stainless steel, alloys thereof, a conductive material may be applied to one or more discrete areas of housing 202 to form electrode 210.

In some examples, electrode 210 may be a component, such as a ring electrode, that is mounted or assembled onto housing 202. Electrode 210 may be electrically coupled to internal circuitry of device 104 via electrically-conductive housing 202 or an electrical conductor when housing 202 is a non-conductive material. In some examples, electrode 210 is located proximate to proximal end 206 of housing 202 and can be referred to as a proximal housing-based electrode. Electrode 210 can also be located at other positions along housing 202, e.g., located proximately to distal end 204 or at other positions along longitudinal axis 212.

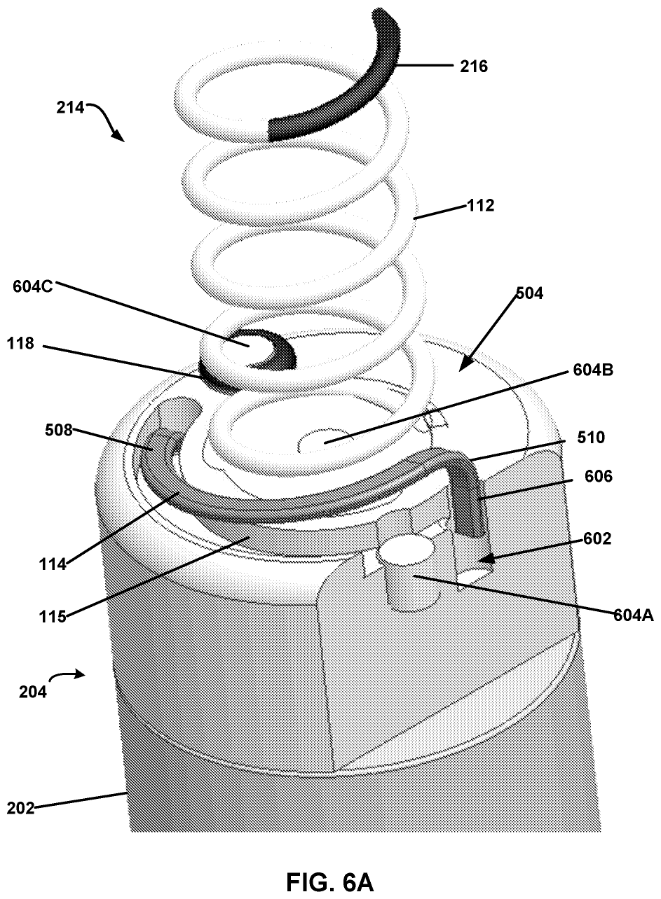

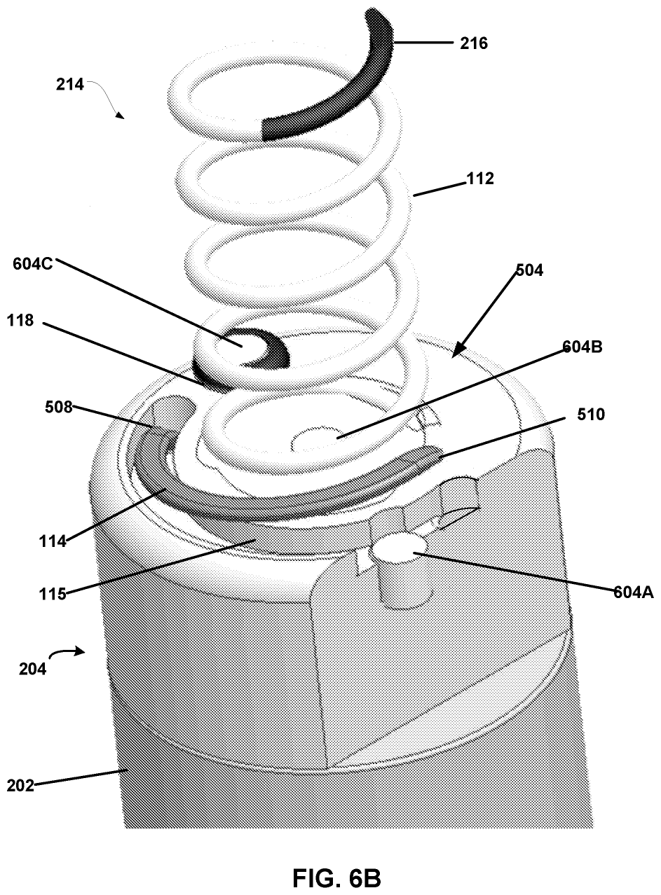

Each of first electrode 112, second electrode 118, and elongated body 114 extends from a first end that is fixedly attached to housing 202 at or near distal end 204, to a second end that, in the example of FIG. 2, is not attached to housing 202 other than via the first end (e.g., is a free end). First electrode 112 may include one or more coatings (e.g., electrically insulative coating(s)) configured to define a first electrically active region 216, or first electrode 112 may otherwise define first electrically active region 216. In some examples, first electrically active region 216 may be more proximate to the second, e.g., distal, end of first electrode 112. In the example of FIG. 2, first electrically active region 216 includes the distal end of electrode 112. Second electrode 118 may include one or more coatings configured to define a second electrically active region 218 on an outer surface of electrode 118. In some examples, as illustrated in FIG. 2, second electrical active region 218 forms a ring around a steroid eluting element or a therapeutic substance dispensing devices, e.g., as discussed in greater detail in with respect to FIGS. 6A and 6B. Second electrode 118 may be a button electrode, a spring electrode, or any other suitable type or shape of electrode.

First and second electrodes 112 and 118 may be formed of an electrically conductive material, such as titanium, platinum, iridium, tantalum, stainless steel or alloys thereof. First and second electrodes 112 and 118 may be coated with an electrically insulating coating, e.g., a parylene, polyurethane, silicone, epoxy, or other insulating coating, to reduce the electrically conductive active surface area of first and second electrodes 112 and 118, and thereby define first and second electrically active regions 216 and 218. Defining first and second electrically active regions 216 and 218 by covering portions with an insulating coating may increase the electrical impedance of first and second electrodes 112 and 118 and thereby reduce the current delivered during a pacing pulse that captures the cardiac tissue. A lower current drain conserves the power source, e.g., one or more rechargeable or non-rechargeable batteries, of device 104.

In some examples, first and second electrodes 112 and 118 may have an electrically conducting material coating on first and second electrically active regions 216 and 218 to define the active regions. For example, first and second electrically active regions 216 and 218 may be coated with titanium nitride (TiN). First and second electrodes 216 and 218 may be made of substantially similar material or may be made of different material from one another.

In the example of FIG. 2, first electrode 112 takes the form of a helix. First electrode 112 may be an elongated body defining a helix. In some examples, a helix is an object having a three-dimensional shape like that of a wire wound uniformly in a single layer around a cylindrical or conical surface or mandrel such that the wire would be in a straight line if the surface were unrolled into a plane. Second electrode 118 is disposed on distal end 204 and may include a button electrode, e.g., as illustrated in FIG. 2, or any other suitable type or shape of electrode. In some examples, device 104 may have a plurality of second electrodes 118 (e.g., two or more second electrodes 118) disposed on distal end 204 of housing 202. The plurality of second electrodes 118 may be equally spaced around a circumference of distal end 204. In some examples, second electrode may be disposed at a user-selected angle away from first end of first electrode 112.

Elongated body 114 includes a ramp portion configured to contact tissue of the patient, e.g., the wall tissue of the chamber in which the device 104 is implanted. The ramp portion of elongated body 114 may be configured as a partial helix, e.g., a helix that does not make a full revolution around a circumference of the cylindrical or conical surface. Elongated body 114 may be formed of an electrically conductive material, such as titanium, platinum, iridium, tantalum, or alloys thereof, and/or of electrically nonconductive material(s). At least portions of elongated body 114 (e.g., the ramp portion of elongated body 114) may be coated with an electrically insulating coating, e.g., a parylene, polyurethane, silicone, epoxy, or other insulating coating. In some examples, elongated body 114 may be formed from a memory metal (e.g., Nitinol, platinum, titanium, MP35N, or the like) and/or a memory polymer (e.g., silicone, polyurethane, polyether ether ketone (PEEK), or the like), or other materials. In some examples, elongated body 114 may be configured to maintain contact with the tissue without significant flexure (e.g., less than or equal 2 millimeters (mm) of flexure).

Elongated body 114 may be disposed over recess 115 and at least partially within recess 115. As a distance between the adjacent tissue and distal end 204 decreases, elongated body 114 may deflect into recess 115, e.g., to maintain contact with the tissue without penetrating the tissue.

Elongated body 114 may be an anti-rotation feature. Elongated body 114 may increase compression of the tissue and/or increase the friction or other fixation force between the tissue and device 104 and/or electrode 112. The increase in fixation force(s) may be sufficient to resist rotation of first electrode 112 by movement of the tissue of heart 102, but may not be sufficient to resist rotation of first electrode 112 by the clinician, e.g., to remove device 104 from heart 102. Elongated body 114 may resist forces of up to about 5 Ounce-force Inches (ozf. in) (e.g., up to about 0.035 Newton meters (Nm)). The amount of force the tissue exerts on first electrode 112 and/or the amount of force elongated body 114 exerts on the tissue may vary based on movement of heart 102, movement of device 104, movement of fluid within heart 102, size of heart 102, or the like.

In some examples, first electrode 112 may include one or more additional anti-rotation features. The additional anti-rotation features may include a shape of first electrode 112, dimensions (e.g., outer diameter, pitch, or the like) of first electrode 112, one or more features disposed on an outer surface of first electrode 112, or the like. The shape and/or dimensions of first electrode 112 may include a geometric shape of first electrode 112, a varying diameter configuration of first electrode 112, a varying pitch configuration of first electrode 112, a waveform configuration of first electrode 112, or any combination herein. The one or more anti-rotation features disposed on first electrode 112 may include, but are not limited to, elongate darts, barbs, or tines. The one or more anti-rotation features may resist rotation of first electrode 112 (e.g., by penetrating the tissue, by increasing the friction between first electrode 112 and the tissue, or the like).

As illustrated in FIG. 2, first electrode 112 may be a right-hand wound helix, and elongated body 114 may be a right-hand wound partial helix, although in other examples the handedness of the electrodes may be switched or the electrodes may each have a different handedness than the each other. Winding elongated body 114 in the same direction as first electrode 112 may reduce the resistance to insertion and/or removal of device 104 by the clinician while increasing the resistance to rotation of device 104 by movement of the tissue and/or movement of heart 102. In the example of FIG. 2, the helix and partial helix defined by first electrode 112 and elongated body 114, respectively, have the same pitch, although they may have different pitches in other examples. In some examples, first electrode 112 has a varying pitch along longitudinal axis 212. In some examples, one or both of electrodes 112 and 114 may have a shape other than helical. For example, first electrode 112 may have a geometrical shape (e.g., a triangular shape, a rectangular shape, a hexagonal shape, an octagonal shape, a lobed shape, or the like). Such a geometrical shape may be equilateral.

First and second electrodes 112 and 118 can also vary in size and shape in order to enhance tissue contact of first and second electrically active regions 216 and 218. For example, first electrodes 112 can have a round cross-section or could be made with a flatter cross-section (e.g., oval or rectangular) based on tissue contact specifications. In some examples, second electrode 118 may have an outer surface that varies in size and shape (e.g., an oval outer surface, an outer surface with a larger diameter, or the like) in order to enhance tissue contact of second electrically active region 218.

The size and shape of first electrode 112 and/or elongated body 114 may be determined at least in part by stiffness requirements. For example, stiffness requirements may vary based on the expected implantation requirements, including the tissue into which the electrodes are implanted or contact, as well as how long device 104 is intended to be implanted.

The distal end of first electrode 112 can have a conical, hemi-spherical, or slanted edge distal tip with a narrow tip diameter, e.g., less than 1 millimeter (mm), for penetrating into and through tissue layers. In some examples, the distal end of first electrode can be a sharpened or angular tip or sharpened or beveled edges, but the degree of sharpness may be constrained to avoid a cutting action that could lead to lateral displacement of the distal end of first electrode 112 and undesired tissue trauma. In some examples, first electrode 112 may have a maximum diameter at its base that interfaces with housing distal end 204. In such examples, the outer diameter of the helix defined by first electrode 112 may decrease from housing distal end 204 to the distal end of first electrode 112. In some examples, the diameter of first electrode 112 may vary from housing distal end 204 to the distal end of first electrode 112. The varying diameter may cause first electrode 112 to resist rotation within the tissue of heart 102.

The outer dimensions of first electrode 112 can be substantially straight and cylindrical, with first electrode 112 being rigid in some examples. In some examples, first electrode 112 and elongated body 114 can have flexibility in lateral directions, being non-rigid to allow some flexing with heart motion. In a relaxed state, when not subjected to any external forces, first electrode 112 can be configured to maintain a distance between first electrically active region 216 and housing distal end 204.

Distal end of first electrode 112 can pierce through one or more tissue layers to position first electrically active region 216 within a desired tissue layer, e.g., the ventricular myocardium 108 or interventricular septum. Accordingly, first electrode 112 extends a distance from housing distal end 204 corresponding to the expected pacing site depth and may have a relatively high compressive strength along its longitudinal axis, which may be substantially similar to or coincident with longitudinal axis 212, to resist bending in a lateral or radial direction when a longitudinal, axial, and/or rotational force is applied, e.g., to the proximal end 206 of housing 202 to advance device 104 into the tissue at target implant region 106. By resisting bending in a lateral or radial direction, first electrode 112 can maintain a spacing between a plurality of windings of first electrode 112 when first electrode 112 is a helix electrode. The spacing may be a pre-determined pitch of first electrode 112 and may vary from distal end 204 to the distal end of first electrode 112. First electrode 112 may be longitudinally non-compressible. First electrode 112 may also be elastically deformable in lateral or radial directions when subjected to lateral or radial forces, however, to allow temporary flexing, e.g., with tissue motion, but returns to its normally straight position when lateral forces diminish. In some examples, when first electrode 112 is not exposed to any external force, or to only a force along its longitudinal axis (substantially similar to or coincident with longitudinal axis 212), first electrode 112 retains a straight, linear configuration as shown.

In some examples, second electrode 118 or electrode 210 may be paired with first electrode 112 for sensing ventricular signals and delivering ventricular pacing pulses. In some examples, second electrode 118 may be paired with electrode 210 or first electrode 112 for sensing atrial signals and delivering pacing pulses to atrial tissue (e.g., to the atrial myocardium) in target implant region 106. In other words, electrode 210 may be paired, at different times, with first electrode 112 and/or second electrode 118 for either ventricular or atrial functionality, respectively, in some examples. In some examples, first and second electrodes 112 and 118 may be paired with each other, with different polarities, for atrial and ventricular functionality.

In some examples, second electrode 118 may be configured as an atrial cathode electrode for delivering pacing pulses to the atrial tissue, e.g. at target implant region 106 in combination with electrode 210. Second electrode 118 and electrode 210 may also be used to sense atrial P-waves for use in controlling atrial pacing pulses (delivered in the absence of a sensed P-wave) and for controlling atrial-synchronized ventricular pacing pulses delivered using first electrode 112 as a cathode and electrode 210 as the return anode.

At distal end 204, device 104 includes a distal fixation assembly 214 including first electrode 112, second electrode 118, elongated body 114, and housing distal end 204. A distal end of first electrode 112 can be configured to rest within a ventricular myocardium of the patient, and second electrode 118 and elongated body 114 can be configured to contact an atrial endocardium of the patient. In some examples, distal fixation assembly 214 can include more or fewer electrodes than two electrodes. In some examples, distal fixation assembly 214 may include one or more second electrodes 118 along housing distal end 204. For example, distal fixation assembly 214 may include two or three electrodes configured for atrial functionality like second electrode 118, and the three electrodes may be substantially similar or different from one another. Spacing between a plurality of second electrodes 118 may be at an equal or unequal distance. Second electrode(s) 118 may be individually selectively coupled to sensing and/or pacing circuitry enclosed by housing 202 for use as an anode with first electrode 112 or as an atrial cathode electrode, or may be electrically common and not individually selectable. In some examples of distal fixation assembly 214, in place of first electrode 112, a fixation element (not shown) of similar shape and mechanical properties may be employed, but without an electrically active region or electrode formed thereon or borne thereby; in such examples, electrically active region 216 can be positioned on a separate member and/or on the housing 202.

Elongated body 114 is configured to flexibly maintain contact between second electrode 118 and the wall tissue of the heart chamber in which device 104 is implanted, e.g., the RA endocardium, despite variations in the tissue surface or in the distance between distal end 204 of housing 202 and the tissue surface, which may occur as the wall tissue moves during the cardiac cycle.

In order to flexibly maintain contact with the wall tissue, elongated body 114 may be flexible and configured to have spring-like properties. In some examples, elongated body 114 may be relatively flexible in some portions and relatively inflexible in other portions. For example, elongated body 114 may be configured to elastically deform, e.g., toward distal end 204 of housing 202, but may be spring biased toward a resting configuration and, when elastically deformed, the spring bias may urge the elongated body 114 away from distal end 204 of housing 202. In this manner, the elastic deformation and spring bias may maintain the elongated body 114 in consistent contact with the wall tissue of the chamber (e.g., the right atrium) in which the device is implanted.

Elongated body 114 may cause second electrode 118 to maintain consistent contact with the wall tissue, e.g., by urging second electrode 118 towards the wall tissue. Consistent contact between second electrode 118 and the wall tissue may improve electrical conductivity and the delivery of electrical signals from second electrode 118 to the wall tissue. In some examples, where device 104 is an implantable pacing device, the consistent contact between second electrode 118 and the wall tissue may reduce and/or maintain a pacing threshold for a chamber (e.g., the right atrium) of heart 102. Elongated body 114 may be disposed at a separation angle about the longitudinal axis 212 away from second electrode 118 to increase the compressive force between second electrode 118 and the wall tissue. In some examples, the separation angle may be an angle between second electrode 118 and the second end (e.g., free end) of elongated body 114. The separation angle may be about 180 degrees, or between about 15 degrees and about 180 degrees. In some examples, a larger separation angle may improve contact in more adverse conditions (e.g., if device 104 is oscillating within heart 102).

As described herein, to flexibly maintain contact may refer to an electrode and/or elongated body being moveable with respect to housing 202. For example, an electrode and/or elongated body may be configured to elastically deform as described above. In some examples, an electrode (e.g., second electrode 118) may additionally be attached to housing 202 by, or may include, a mechanism, such as a spring or joint, that allows relative motion of the electrode to housing 202. In such examples, the electrode need not itself be deformable.

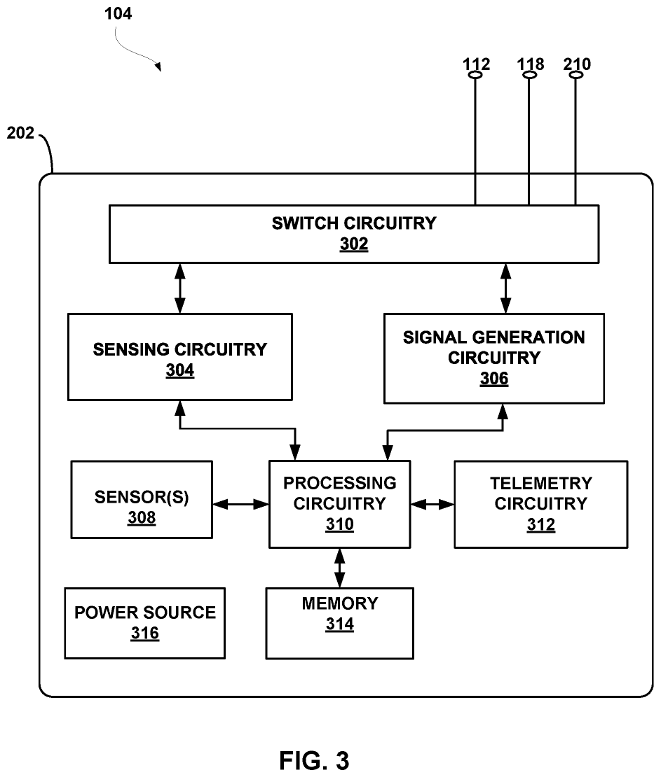

FIG. 3 is a functional block diagram illustrating an example configuration of device 104. As illustrated in FIG. 3, device 104 include electrodes 112 and 118, which may be configured as described with respect to FIGS. 1 and 2. For example, as described with respect to FIGS. 1 and 2, first electrode 112 may be configured to extend from distal end 204 of housing 202 and may penetrate through the wall tissue of a first chamber (e.g., the RA) into wall tissue of a second chamber (e.g., the LV). Second electrode 118 extends from distal end 204 of housing 202 and may be configured to flexibly maintain contact with the wall tissue of the first chamber without penetration of the wall tissue of the first chamber by the second electrode. Second electrode 118 may flexibly maintain contact in this manner by virtue of being spring biased distally away from housing 202, and/or due to the action of elongated body 114, or otherwise.

In the example shown in FIG. 3, device 104 includes switch circuitry 302. sensing circuitry 304, signal generation circuitry 306, sensor(s) 308, processing circuitry 310, telemetry circuitry 312, memory 314, and power source 316. The various circuitry may be, or include, programmable or fixed function circuitry configured to perform the functions attributed to respective circuitry. Memory 314 may store computer-readable instructions that, when executed by processing circuitry 310, cause device 104 to perform various functions. Memory 314 may be a storage device or other non-transitory medium. The components of device 104 illustrated in FIG. 3 may be housed within housing 202.

Signal generation circuitry 306 generates electrical stimulation signals, e.g., cardiac pacing pulses. Switch circuitry 302 is coupled to electrodes 112, 118, and 210, may include one or more switch arrays, one or more multiplexers, one or more switches (e.g., a switch matrix or other collection of switches), one or more transistors, or other electrical circuitry. Switch circuitry 302 is configured to direct stimulation signals from signal generation circuitry 306 to a selected combination of electrodes 112, 118, and 210, having selected polarities, e.g., to selectively deliver pacing pulses to the RA, ventricles, or interventricular septum of heart 102. For example, in order to pace one or both of the ventricles, switch circuitry 302 may couple first electrode 112, which has penetrated to wall tissue of a ventricle or the intraventricular septum, to signal generation circuitry 306 as a cathode, and one or both of second electrode 118 or electrode 210 to signal generation circuitry 306 as an anode. As another example, in order to pace the RA, switch circuitry 302 may couple second electrode 118, which flexibly maintains contact with the RA endocardium, to signal generation circuitry 306 as a cathode, and one or both of first electrode 112 or electrode 210 to signal generation circuitry 306 as an anode.

Switch circuitry 302 may also selectively couple sensing circuitry 304 to selected combinations of electrodes 112, 118, and 210, e.g., to selectively sense the electrical activity of either the RA or ventricles of heart 102. Sensing circuitry 304 may include filters, amplifiers, analog-to-digital converters, or other circuitry configured to sense cardiac electrical signals via electrodes 112, 118, and/or 210. For example, switch circuitry 302 may couple each of first electrode 112 and second electrode 118 (in combination with electrode 210) to respective sensing channels provided by sensing circuitry 304 to respectively sense either ventricular or atrial cardiac electrical signals. In some examples, sensing circuitry 304 is configured to detect events, e.g., depolarizations, within the cardiac electrical signals, and provide indications thereof to processing circuitry 310. In this manner, processing circuitry 310 may determine the timing of atrial and ventricular depolarizations, and control the delivery of cardiac pacing, e.g., AV synchronized cardiac pacing, based thereon. Processing circuitry 310 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 310 herein may be embodied as firmware, hardware, software or any combination thereof.

Sensor(s) 308 may include one or more sensing elements that transduce patient physiological activity to an electrical signal to sense values of a respective patient parameter. Sensor(s) 308 may include one or more accelerometers, optical sensors, chemical sensors, temperature sensors, pressure sensors, or any other types of sensors. Sensor(s) 308 may output patient parameter values that may be used as feedback to control sensing and delivery of therapy by device 104.

Telemetry circuitry 312 supports wireless communication between device 104 and an external programmer (not shown in FIG. 3) or another computing device under the control of processing circuitry 310. Processing circuitry 310 of device 104 may receive, as updates to operational parameters from the computing device, and provide collected data, e.g., sensed heart activity or other patient parameters, via telemetry circuitry 312. Telemetry circuitry 312 may accomplish communication by radiofrequency (RF) communication techniques, e.g., via an antenna (not shown).

Power source 316 delivers operating power to various components of device 104. Power source 316 may include a rechargeable or non-rechargeable battery and a power generation circuit to produce the operating power. Recharging may be accomplished through proximal inductive interaction between an external charger and an inductive charging coil within device 104.

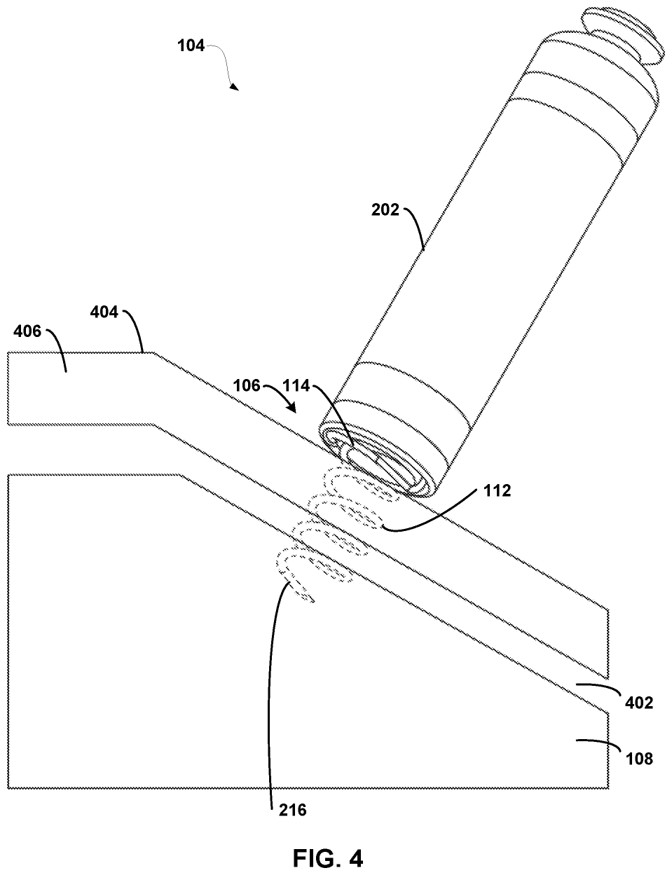

FIG. 4 is a conceptual diagram of device 104 implanted at target implant region 106. First electrode 112 may be inserted (e.g., in a manner similar to rotating and advancing a threaded screw) such that tissue becomes engaged with the helix of first electrode 112. As first electrode 112 becomes engaged with tissue, first electrode 112 pierces into the tissue at target implant region 106 and advances through atrial myocardium 406 and central fibrous body 402 to position first electrically active region 216 in ventricular myocardium 108 as shown in FIG. 4. In some examples, first electrode 112 penetrates into the interventricular septum. In some examples, first electrode 112 docs not perforate entirely through the ventricular endocardial or epicardial surface.

In some examples, manual pressure applied to the housing proximal end 206, e.g., via an advancement tool, provides the longitudinal force to pierce the cardiac tissue at target implant region 106. In some examples, actuation of an advancement tool rotates device 104 and first electrode 112 configured as a helix about longitudinal axis 212. The rotation of the helix about the longitudinal axis 212 advances first electrode 112 through atrial myocardium 406 and central fibrous body 402 to position first electrically active region 216 in ventricular myocardium 108 as shown in FIG. 4.

As first electrode 112 advances into the tissue, the distance between elongated body 114 and atrial endocardium 404 decreases until elongated body 114 contacts, and may press against, the surface of atrial endocardium 404. Elongated body 114 may press against the surface of atrial endocardium 404 and compress the wall tissue. The compression of the wall tissue may increase friction between elongated body 114 and the wall tissue and prevent rotation of first electrode 112 due to movement of tissue of heart 102 (e.g., movement of ventricular myocardium 108, atrial myocardium 406, central fibrous body 402, or the like). Elongated body 114 pressing against heart tissue may cause heart tissue to become engaged with second electrically active region 218 of second electrode 118 disposed a separation angle away from elongated body 114. Second electrode 118 is held in contact with atrial endocardium 404 by first electrode 112 and elongated body 114. Retraction of second electrode 118 from the surface of atrial endocardium 404 may be prevented by first electrode 112 and the forced exerted by elongated body 114 upon atrial endocardium 404. Elongated body 114 is also configured, as described herein, to flexibly maintain contact with atrial endocardium 404. In some examples, elongated body 114 is elastically deformable toward distal end 204 of housing 202 and into recess 115 and has a spring bias urging elongated body 114 distally from distal end 204.

Elongated body 114 can be the sole anti-rotation feature of device 104 in some examples. In some examples, device 104 may have one or more additional anti-rotation features, e.g., defined and/or disposed on first electrode 112. The distance by which first electrode 112 extends from housing 202 can be selected so first electrically active region 216 reaches an appropriate depth in the tissue layers to reach the targeted pacing and sensing site, in this case in ventricular myocardium 108, without puncturing all the way through into an adjacent cardiac chamber.

Target implant region 106 in some pacing applications is along atrial endocardium 404, substantially inferior to the AV node and bundle of His. First electrode 112 can have a length that penetrates through atrial endocardium 404 in target implant region 106, through the central fibrous body 402 and into ventricular myocardium 108 without perforating through the ventricular endocardial surface. In some examples, when the full length of first electrode 112 is fully advanced into target implant region 106, first electrically active region 216 rests within ventricular myocardium 108 and second electrode 118 is positioned in intimate contact with atrial endocardium 404. First electrode 112 may extend from housing distal end 204 approximately 3 mm to 12 mm in various examples. In some examples, first electrode 112 may extend a distance from housing 202 of at least 3 millimeters (mm), at least 3 mm but less than 20 mm, less than 15 mm, less than 10 mm, or less than 8 mm in various examples. The diameter of first electrode 112 may be less than 2 mm and may be 1 mm or less, or even 0.6 mm or less.

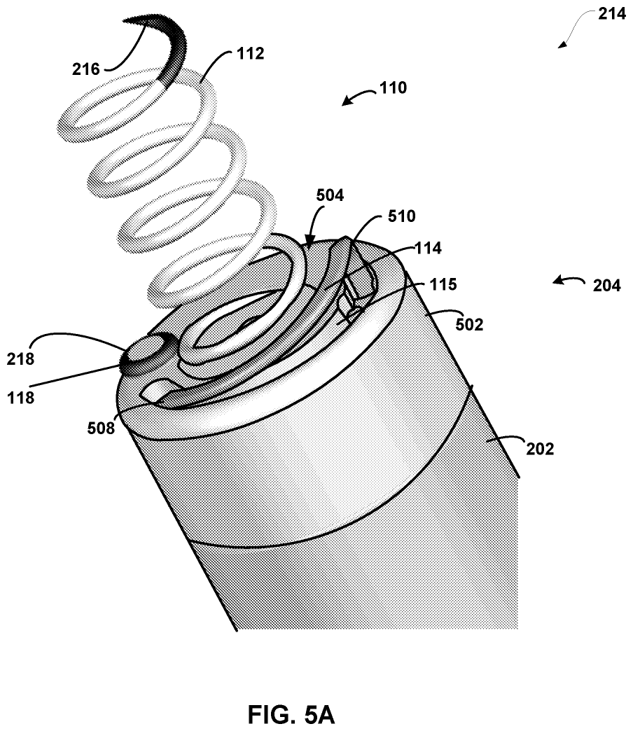

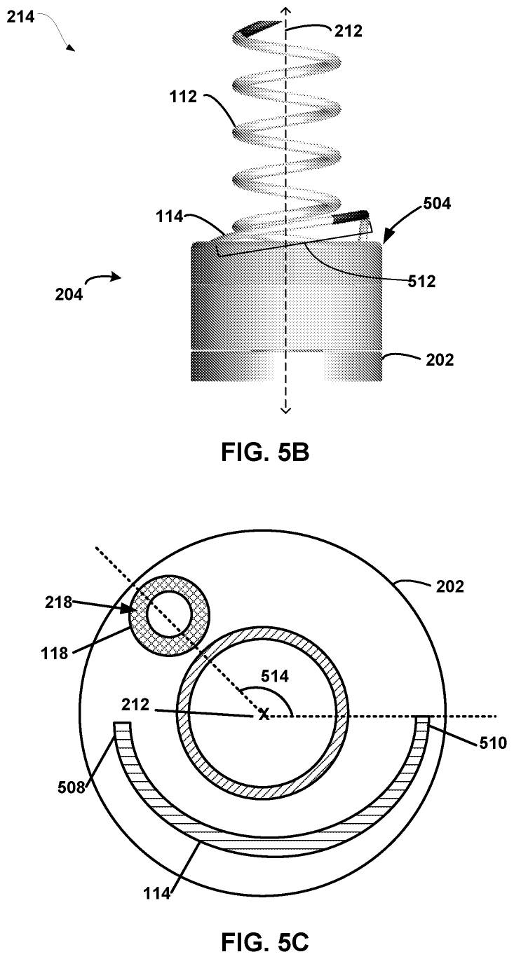

FIGS. 5A, 5B, and 5C are partial views of the device of FIGS. 1-4 from different perspectives, in accordance with one or more aspects of this disclosure. FIG. 5A is a partial view of distal end 110 of device 104 including distal fixation assembly 214. Housing 202 includes a header 502. In some examples, header 502 may be separate or integral with housing 202 and can be made of the same or different materials as housing 202. Housing distal end 204, e.g., header 502 or face 505 of distal end 204, defines a recess 115 (e.g., a recessed channel) to receive at least a portion of elongated body 114 as it is elastically deformed toward housing 202. Second electrically active region 218 of second electrode 118 disposed on distal end 204 can maintain contact with the tissue surface when elongated body 114 is partially or fully deformed into recess 115.

In some examples, as illustrated in FIG. 5A, distal end 204 of housing 202 includes a peripheral region, e.g., around first electrode 112. Elongated body 114 and second electrode 118 may be disposed in the peripheral region of distal end 204.

In some examples, elongated body 114 and second electrode 118 can maintain contact with tissue as the extent of deformation of elongated body 114 toward housing 202 varies. Elongated body 114 may be spring biased to an undeformed position, and deformation of elongated body 114 proximally toward distal end 204 of housing 202 may result in a spring force directed distally from housing 202 that urges elongated body 114 and second electrode 118 disposed a separation angle away from elongated body 114, and more particularly second electrically active region 218 of second electrode 118, against cardiac tissue. As elongated body 114 urges a portion of housing 202 distally away from the adjacent heart tissue, first electrode 112 can act as a fulcrum or pivot whereby second electrode 118 is urged in the proximal direction toward the adjacent heart tissue. Deformation of elongated body 114 may vary with the motion of the heart. Because, at least in part, of the ability of the deformation of elongated body 114 to vary, e.g., during the cardiac cycle, second electrode 118 can maintain consistent contact with the tissue and provide pacing to the heart, e.g., due to the separation angle between second electrode 118 and elongated body 114.

Elongated body 114 may extend from a first end 508 to a second end 510. First end 508 may be connected to distal end 204 of housing 202. In some examples, as illustrated in FIG. 5A, second end 510 may be connected to distal end 204 or may be a free end (e.g., free-floating or cantilever). In some examples, second end 510 may include a protrusion extending proximally towards distal end 204, e.g., as illustrated and described in greater detail in FIG. 6A. Second end 510 and/or the protrusion may be free-floating to facilitate removal of device 104 from the tissue, e.g., by allowing removal of elongated body 114 from overgrown tissue in recess 115.

FIG. 5B is a conceptual diagram of a side view of device 104 of FIG. 5A, in accordance with one or more aspects of this disclosure. FIG. 5C is a conceptual diagram of a top-down view of device 104, in accordance with one or more aspects of this disclosure.

As illustrated in FIG. 5B, elongated body 114 may include ramp portion 512 extending distally away from distal end 204. Recess 115 within housing 202 may include a corresponding indentation such that ramp portion 512 may be fully disposed within recess 115. In some examples, a proximal end of ramp portion 512 may engage with tissue and increase resistance between elongated body 114 and tissue, thereby cause device 104 to resist rotation.

As illustrated in FIG. 5C, second electrode 118 may be separated from elongated body 114 by separation angle 514 about longitudinal axis 212. In some examples, as illustrated in FIG. 5C, angle 514 is an angle between second electrode 118 (e.g., a central axis of second electrode 118) and second end 510 of elongated body 114. Angle 514 may be about 180 degrees, or between about 15 degrees and about 180 degrees. In some examples, where device 104 include two or more second electrodes 118, angle 514 may be between second end 510 of elongated body 114 and one or more of second electrodes 118. In some examples, each of a plurality of second electrodes 118 may be separated from second end 510 of elongated body 114 by at least angle 514.

FIG. 6A is a conceptual diagram illustrating a partial view of device 104 of FIGS. 1-5C with a cutout illustrating recess 115. FIG. 6B is a conceptual diagram illustrating a partial view of another example device 104 of FIGS. 1-5C with a cutout illustrating recess 115. FIG. 6A illustrates an example elongated body 114 with protrusion 606 disposed at second end 510 and configured to be disposed within recess hole 602 in recess 115. FIG. 6B illustrates another example elongated body 114 without protrusion 606 and recess hole 602 in recess 115.

As illustrated in FIGS. 6A and 6B, first end 508 of elongated body 114 is attached to header 502 and is connected (e.g., electrically) to a feedthrough. As illustrated in FIG. 6A, at the opposite end of elongated body 114, second end 510 of elongated body 114 is bent back towards device 104 and forms protrusion 606. Protrusion 606 may be able to move into recess hole 602 within recess 115, e.g., as elongated body 114 is deformed towards distal end 204 of housing 202. Second electrode 118 may maintain contact with tissue while protrusion 606 is pushed into recess hole 602 due to deformation of elongated body 114 with heart motion.

In some examples, during extraction of device 104 from heart 102 by the clinician, protrusion 606 may deflect distal to distal end 204 (e.g., due to spring bias of elongated body 114 and/or overgrown tissue within recess 115 and/or recess hole 602). The overgrown tissue within recess 115 and/or recess hole 602 may be due to natural growth of tissue, e.g., around a foreign object. Protrusion 606 may deflect distal to distal end 204 and allow the overgrown tissue to exit recess 115 and/or recess hole 602, thereby allowing removal of device 104 without requiring the clinician to remove the overgrown tissue. In some examples, as illustrated in FIG. 6B, elongated body 114 may not include protrusion 606 at second end 510 and may further facilitate the removal of the device 104 and/or overgrown tissue.

As discussed herein, first electrode 112 and elongated body 114 can have different handed helical shapes. For example, the helix of first electrode 112 and a partial helix defined by elongated body 114 can both be right-handed. First electrode 112 can be inserted, e.g., in a manner similar to rotating and advancing a threaded screw, such that tissue becomes engaged with the helix of first electrode 112. As the distance between elongated body 114 and the tissue decreases due to right-hand rotation of first electrode 112, the tissue will gradually contact ramp portion 512 (illustrated in FIG. 5C) of elongated body 114 similar to advancing along a ramp, and the ramp-shape of ramp portion 512 will gradually deform, e.g., compress, toward housing 202.

In some examples, first electrode 112 includes a helix with a first pitch, and ramp portion 114 of elongated body 114 is a partial helix with a second pitch. A first pitch of the helix of first electrode 112 can be the same, substantially similar, or different than the second pitch of the partial helix of elongated body 114. In some examples, elongated body 114 can be more peripheral than first electrode 112 relative to longitudinal axis 212. In some examples, first electrode 112 resides in an inner space defined by elongated body 114 and is approximately concentric with elongated body 114.

Inflammation of patient tissue may result from interaction with device 104. For example, penetration of tissue by a first electrode and/or contact between tissue and the second electrode may result in inflammation of the tissue. Inflammation of patient tissue proximate to electrodes may result in higher thresholds for stimulation delivered to the tissue to activate, or capture, the tissue. Higher capture thresholds may, in turn, increase the consumption of a power source of the IMD 104 associated with delivery of the stimulation.

In some examples, an IMD as described herein, such as IMDs 104 may include one or more steroid eluting elements 604A-C (collectively referred to as “steroid eluting elements 604”), e.g., disposed on distal end 204. The steroid may mitigate inflammation of patient tissue resulting from interaction with the IMD. Steroid eluting elements 604 may be configured to elute one or more steroids to tissue in proximity to elements 604 over time. In some examples, steroid eluting elements 604 comprise one or more monolithic controlled release devices (MCRDs). In some examples, steroid eluting elements 604 may be therapeutic substance dispensing devices.

In some examples, an IMD includes one or both of a first steroid eluting element 604A configured to elute one or more steroids to tissue proximate to first electrode 112, and a second steroid eluting element 604B configured to elute one or more steroids to tissue proximate to elongated body 114. IMD 104 includes steroid eluting elements 604 at distal end 204 of housing 202, e.g., included with, attached to, or formed on header 502. Steroid eluting elements 604 may elute one or more steroids to wall tissue proximate to elongated body 114. In some examples, as illustrated in FIGS. 6A and 6B, second steroid eluting element 604B may be disposed within recess 115. In some examples, one or more steroid eluting elements 604 may be disposed on second electrode 118 (e.g., on second electrically active region 218).

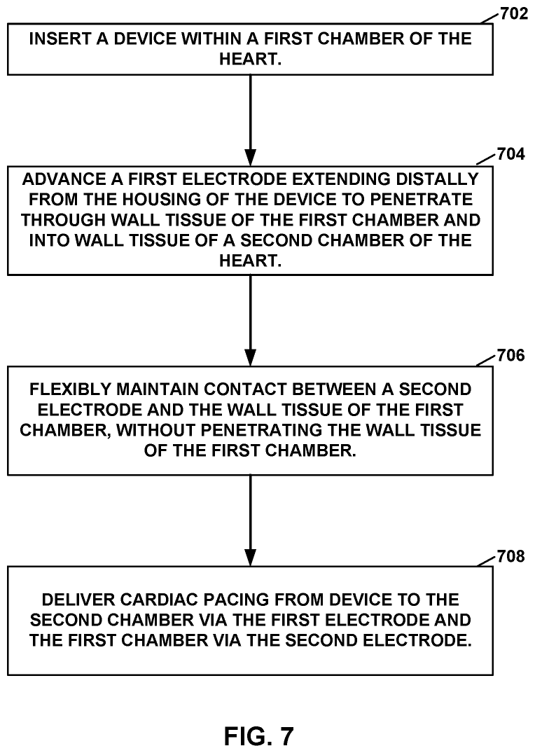

FIG. 7 is a flow diagram illustrating an example process for deploying an example device. The technique of FIG. 7 will be described with concurrent reference to device 104 (FIG. 1) although a person having ordinary skill in the art will understand that the technique may be performed in reference to another implantable medical lead or other medical device.

A clinician may insert device 104 within a single first chamber of the heart 102 (702). The first chamber of heart 102 may be the right atrium, left atrium, the right ventricle, or the left ventricle. The clinician may insert device 104 into the first chamber via delivery tool connected to device 104 (e.g., to delivery tool interface member 208). The clinician may advance first electrode 112 extending distally from housing 202 of device 104 to penetrate through wall tissue of the first chamber and into wall tissue of a second chamber of heart 102 (704). In some examples, advancing first electrode 112 includes positioning a distal end of first electrode 112 (e.g., a first electrically active region 216) within a ventricular myocardium 108 of the patient. The clinician may advance first electrode 112 by rotating device 104 clockwise or counter-clockwise within the first chamber, depending on how first electrode 112 is wound.

The clinician may cause device 104 to establish and maintain contact between elongated body 114 and the wall tissue of the first chamber, without penetrating the wall tissue of the first chamber (706). In some examples, elongated body 114 may flexibly maintain contact with the wall tissue. In some examples, flexibly maintaining contact with the wall tissue of the first chamber with elongated body 114 includes contacting atrial endocardium 402 of the patient. In some examples, flexibly maintaining contact with the wall tissue of the first chamber with elongated body 114 includes deforming elongated body 114 toward elongated housing 202 by the wall tissue of the first chamber as a distance between the distal end of elongated housing 202 and the wall tissue of the first chamber decreases. In some examples, recess 115 receives at least a portion of elongated body 114 as it is elastically deformed back toward elongated housing 202. In some examples, a spring bias of elongated body 114 urges elongated body 114 away from housing 202 and into consistent contact with the wall tissue of the first chamber. Elongated body 114 may cause second electrode 118 disposed on distal end 204 of housing 202 to maintain consistent contact with the wall tissue of the first chamber (e.g., atrial endocardium 402 of patient), e.g., due to at least in part the spring bias of elongated body 114. Second electrode 118 may be disposed at a separation angle 514 about longitudinal axis 212 away from elongated body 114 (e.g., from second end 510 of elongated body 114).

While device 104 is implanted within the cardiac tissue, the one or more anti-rotation features defined resist rotation of device 104 due to movement of the cardiac tissue. The one or more anti-rotation features may prevent movement of first electrode 112 away from wall tissue of the second chamber of heart 102 (e.g., ventricular myocardium 108 of the patient). The one or more anti-rotation features may also prevent dislodgement of device 104 from within wall tissue of the first chamber of heart 102. The one or more anti-rotation features may include elongated body 114 maintaining contact with the wall tissue of the first chamber. Elongated body 114 may increase compression of the wall tissue which causes device 104 to resist rotation due to movement of the wall tissue.

The clinician may deliver cardiac pacing from device 104 to the second chamber via first electrode 112 and to the first chamber via second electrode 118 (708). Device 104 may deliver cardiac pacing to the first chamber and/or the second chamber via first electrode 112, second electrode 118, and/or one or more other electrodes of device 104 (e.g., electrode 210).

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

In addition, it should be noted that system described herein may not be limited to treatment of a human patient. In alternative examples, the system may be implemented in non-human patients, e.g., primates, canines, equines, pigs, and felines. These other animals may undergo clinical or research therapies that may benefit from the subject matter of this disclosure.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Example 1: a fixation device comprising: a first elongated body extending distally from a distal end of an implantable medical device, the first elongated body comprising: a helix having one or more coils, wherein a distal end of the helix is configured to penetrate into tissue of a patient; and a second elongated body extending distally from the distal end of the implantable medical device, wherein the second elongated body is separate from the first elongated body, wherein the second elongated body is connected to the implantable medical device at a first location and when unstressed is furthest distally from the implantable medical device at a second location, wherein the second elongated body is configured to flexibly maintain contact with the tissue without penetrating the tissue, and wherein the second elongated body is disposed on the distal end of the implantable medical device at a separation angle away from an electrode disposed on the distal end of the implantable medical device, wherein the separation angle comprises an angle between the electrode and the second location of the second elongated body.

Example 2: the device of example 1, wherein the first elongated body comprises a first electrode, and wherein the electrode disposed on the distal end of the implantable medical device comprises a second electrode.

Example 3: the device of any of examples 1 and 2, wherein the implantable medical device comprises an implantable pacemaker configured to be implanted wholly within a first chamber of a heart of the patient.

Example 4: the device of any of examples 1-3, wherein the implantable medical device is configured to be implanted within a first chamber of a heart of the patient, wherein the first elongated body is configured to penetrate wall tissue of a second chamber of the heart, and wherein the second elongated body is configured to flexibly maintain contact between the electrode and wall tissue of the first chamber of the heart.

Example 5: the device of example 4, wherein a distal end of the first elongated body is configured to penetrate into a ventricular myocardium of the patient, and wherein the second elongated body is configured to flexible maintain contact between the electrode and an atrial endocardium of the patient.

Example 6: the device of any of examples 1-5, wherein the second elongated body is configured as a partial helix.

Example 7: the device of example 6, wherein the partial helix is wound in a same direction as the helix of the first elongated body.

Example 8: the device of example 7, wherein the partial helix and the helix of the first elongated body are wound in a counter-clockwise direction.

Example 9: the device of any of examples 1-8, wherein the distal end of the implantable medical device defines a first recess configured to retain at least a portion of the second elongated body as the second elongated body flexibly maintains contact with the tissue.

Example 10: the device of example 9, wherein the second elongated body further comprises a protrusion disposed at the second location of the second elongated body, and wherein the distal end of the implantable medical device further defines a second recess within the first recess, wherein the second recess is configured to retain the protrusion of the second elongated body.

Example 11: the device of any of examples 9 and 10, wherein the distal end of the implantable medical device further comprises a therapeutic substance dispensing device disposed within the first recess.

Example 12: the device of example 11, wherein the therapeutic substance dispensing device comprises a monolithic controlled release device.

Example 13: the device of any of examples 11 and 12, wherein the implantable medical device further comprises a plurality of therapeutic substance dispensing devices disposed on the distal end of the implantable medical device.

Example 14: the device of any of examples 1-13, wherein the second location of the second elongated body comprises a distal-most end of the second elongated body.

Example 15: the device of any of examples 1-14, wherein the separation angle is 15 degrees or greater.

Example 16: the device of any of examples 1-15, wherein the separation angle is 180 degrees.

Example 17: the device of any of examples 1-16, wherein the second elongated body is configured to cause the electrode to maintain contact against the tissue.

Example 18: the device of any of examples 1-17, wherein the first elongated body and the second elongated body comprises a metallic alloy.

Example 19: the device of example 18, wherein the second elongated body comprises a Nickel-Titanium alloy.

Example 20: the device of any of examples 1-19, wherein the first elongated body resides in an inner space defined by the second elongated body and is substantially concentric with the second elongated body.

Example 21: the device of any of examples 1-20, wherein the second elongated body is configured to be elastically deformed towards the distal end of the implantable medical device by the tissue as a distance between the distal end and the tissue decreases to maintain contact with the tissue without penetration of the tissue.

Example 22: the device of any of examples 1-21, wherein the second electrode is a button electrode.

Example 23: the device of any of examples 1-22, wherein the second elongated body is disposed on a peripheral region of the distal end of the implantable medical device.

Example 24: the device of any of examples 1-23, wherein the second electrode is disposed on a peripheral region of the distal end of the implantable medical device.

Example 25: the device of any of examples 1-24, wherein the first elongated body is disposed on the distal end of the implantable medical device between the second elongated body and the second electrode.

Example 26: the device of any of examples 1-25, wherein the second elongated body is configured to urge the second electrode towards the tissue of the patient.

Example 27: The device of example 26, wherein the second elongated body is configured to urge a first portion of the distal end of the implantable medical device away from the tissue of the patient while the second elongated body is urging the second electrode towards the tissue of the patient.

Example 28: a device comprising: an elongated housing that extends from a proximal end to a distal end, the elongated housing configured to be implanted wholly within a first chamber of a heart, the first chamber of the heart having wall tissue; a first electrode extending distally from the distal end of the elongated housing, the first electrode comprising: a first elongated body defining a helix, wherein the helix is configured to penetrate into wall tissue of a second chamber of the heart that is separate from the first chamber of the heart; a second electrode disposed on the distal end of the elongated housing; a second elongated body extending distally from the distal end of the elongated housing, wherein the second elongated body is connected to the distal end at a first location and when unstressed is furthest distally from the distal end of the elongated housing at a second location, wherein the second elongated body is separate from the first electrode and the second electrode, wherein the second elongated body is configured to maintain contact between the second electrode and the wall tissue of the first chamber without penetrate of the wall tissue of the first chamber by the second elongated body, and wherein the second elongated body is disposed on the distal end at a separation angle away from an electrode disposed on the distal end of the implantable medical device, wherein the separation angle comprises an angle between the electrode and the second location of the second elongated body; and signal generation circuitry within the elongated housing, the signal generation circuitry being coupled to the first electrode and the second electrode, wherein the signal generation circuitry is configured to deliver cardiac pacing to the second chamber via the first electrode and the first chamber via the second electrode.

Example 29: the device of example 28, wherein the second electrode comprises a button electrode.

Example 30: the device of any of examples 28 and 29, wherein the second elongated body is configured to resist rotation of the first electrode due to movement of the wall tissue of the first chamber.

Example 31: the device of any of examples 28-30, wherein the first electrode is configured to penetrate into a ventricular myocardium of the patient, and wherein the second elongated body is configured to flexibly maintain contact between the second electrode and an atrial endocardium of the patient.

Example 32: the device of any of examples 28-31, wherein the second elongated body is configured as a partial helix.

Example 33: the device of example 32, wherein the partial helix is wound in a same direction as the helix of the first elongated body.

Example 34: the device of example 33, wherein the partial helix and the helix of the first elongated body are wound in a counter-clockwise direction.

Example 35: the device of any of examples 28-34, wherein the distal end of the elongated housing defines a first recess configured to retain at least a portion of the second elongated body as the second elongated body flexibly maintains contact between the second electrode and the wall tissue of the first chamber.

Example 36: the device of example 35, wherein the second elongated body further comprises a protrusion disposed at the second location of the second elongated body, wherein the distal end of the elongated housing further defines a second recess within the first recess, and wherein the second recess is configured to retain the protrusion of the second elongated body.

Example 37: the device of any of examples 35 and 36, wherein the distal end of the elongated housing further comprises a plurality of therapeutic substance dispensing devices.

Example 38: the device of example 37, wherein one or more of the plurality of therapeutic substance dispensing devices is disposed within the first recess.

Example 39: the device of any of examples 28-38, wherein the second location of the second elongated body comprises a distal-most end of the second elongated body.

Example 40: the device of example 39, wherein the separation angle is 15 degrees or greater.

Example 41: the device of any of examples 28-40, wherein the separation angle is 180 degrees.

Example 42: the device of any of examples 28-41, wherein the second elongated body is configured to cause the second electrode to maintain contact with the wall tissue of the first chamber.

Example 43: the device of any of examples 28-42, wherein the first elongated body and the second elongated body comprises a metallic alloy.

Example 44: the device of example 43, wherein the second elongated body comprises a Nickel-Titanium alloy.

Example 45: the device of any of examples 28-44, wherein the first elongated body resides in an inner space defined by the second elongated body and is substantially concentric with the second elongated body.

Example 46: the device of any of examples 28-45, wherein the second elongated body is configured to be elastically deformed towards the distal end of the elongated housing by the wall tissue of the first chamber as a distance between the distal end and the wall tissue of the first chamber decreases to maintain contact between the second electrode and the wall tissue of the first chamber without penetration of the wall tissue of the first chamber.

Example 47: the device of any of examples 28-46, wherein the second elongated body is disposed on a peripheral region of the distal end of the elongated housing.

Example 48: the device of any of examples 28-47, wherein the second electrode is disposed on a peripheral region of the distal end of the elongated housing.

Example 49: the device of any of examples 28-48, wherein the first elongated body is disposed on the distal end of the elongated housing between the second elongated body and the second electrode.

Example 50: the device of any of examples 1-49, wherein the second elongated body is configured to urge the second electrode towards the tissue of the patient.

Example 51: the device of example 50, wherein the second elongated body is configured to urge a first portion of the distal end of the implantable medical device away from the tissue of the patient while the second elongated body is urging the second electrode towards the tissue of the patient.

Example 52: a method comprising: delivering cardiac pacing from a device to a heart, wherein the device comprises: an elongated housing extending from a proximal end to a distal end, the elongated housing configured to be implanted wholly within a first chamber of the heart, the first chamber of the heart having wall tissue; a first electrode extending distally from the distal end of the elongated housing, the first electrode comprising: a first elongated body defining a helix; a second electrode disposed on the distal end of the elongated housing; a second elongated body extending from the distal end of the elongated housing, wherein the second elongated body is connected to the distal end at a first location and when unstressed is furthest distally from the distal end of the elongated housing at a second location, wherein the second elongated body is separate from the first electrode and the second electrode, and wherein the second elongated body is configured to flexibly maintain contact between the second electrode and the wall tissue of the first chamber without penetration of the wall tissue of the first chamber by the second electrode or the second elongated body, wherein the second elongated body is disposed on the distal end at a separation angle away from an electrode disposed on the distal end of the implantable medical device, wherein the separation angle comprises an angle between the electrode and the second location of the second elongated body; and signal generation circuitry within the elongated housing, the signal generation circuitry being coupled to the first electrode and the second electrode, wherein delivering the cardiac pacing comprises: delivering cardiac pacing to a second chamber via the first electrode; and delivering cardiac pacing to the first chamber via the second electrode.

Example 53: The method of example 52, wherein the second electrode comprises a button electrode.

Example 54: The method of any of examples 52 and 53, wherein the second elongated body is configured to resist rotation of the first electrode due to movement of the wall tissue of the first chamber.

Example 55: the method of any of examples 52-54, further comprising: penetrating a ventricular myocardium of the patient with a first electrode of the device; and placing the second electrode in contact with an atrial endocardium of the patient.

Example 56: the method of any of examples 52-55, wherein the second elongated body is configured as a partial helix.

Example 57: the method of example 56, wherein the partial helix is wound in a same direction as the helix of the first elongated body.

Example 58: the method of example 57, wherein the partial helix and the helix of the first elongated body are wound in a counter-clockwise direction.

Example 59: the method of any of examples 52-58, wherein the distal end of the elongated housing defines a first recess configured to retain at least a portion of the second elongated body as the second elongated body flexibly maintains contact between the second electrode and the wall tissue of the first chamber.

Example 60: the method of example 59, wherein the second elongated body further comprises a protrusion disposed at a second location of the second elongated body, wherein the distal end of the elongated housing further defines a second recess within the first recess, and wherein the second recess is configured to retain the protrusion of the second elongated body.

Example 61: the method of any of examples 59 and 60, wherein the distal end of the elongated housing further comprises a plurality of therapeutic substance dispensing devices.

Example 62: the method of any of examples 52-61, wherein the second location of the second elongated body comprises a distal-most end of the second elongated body.

Example 63: the method of example 62, wherein the separation angle is 15 degrees or greater.

Example 64: the method of any of examples 52-63, wherein the separation angle is 180 degrees.

Example 65: the method of any of examples 52-64, wherein the second elongated body is configured to cause the second electrode to maintain contact with the wall tissue of the first chamber.

Example 66: the method of any of examples 52-65, wherein the first elongated body and the second elongated body comprises a metallic alloy.

Example 67: the method of example 66, wherein the second elongated body comprises a Nickel-Titanium alloy.

Example 68: the method of any of examples 52-67, wherein the first elongated body resides in an inner space defined by the second elongated body and is substantially concentric with the second elongated body.

Example 69: the method of any of examples 52-68, wherein the second elongated body is configured to be elastically deformed towards the distal end of the elongated housing by the wall tissue of the first chamber as a distance between the distal end and the wall tissue of the first chamber decreases to maintain contact between the second electrode and the wall tissue of the first chamber without penetration of the wall tissue of the first chamber.

Example 70: the method of any of examples 52-69, wherein the second elongated body is disposed on a peripheral region of the distal end of the implantable medical device.

Example 71: the method of any of examples 52-70, wherein the second electrode is disposed on a peripheral region of the distal end of the elongated housing.

Example 72: the method of any of examples 52-71, wherein the first elongated body is disposed on the distal end of the elongated housing between the second elongated body and the second electrode.

Example 73: the method of any of examples 52-72, wherein the second elongated body is configured to urge the second electrode towards the tissue of the patient.

Example 74: the method of example 73, wherein the second elongated body is configured to urge a first portion of the distal end of the implantable medical device away from the tissue of the patient while the second elongated body is urging the second electrode towards the tissue of the patient.

Example 75: a fixation device comprising: a first elongated body extending distally from a distal end of an implantable medical device, the first elongated body comprising: a helix having one or more coils, wherein a distal end of the helix is configured to penetrate into tissue of a patient; and a second elongated body extending distally from the distal end of the implantable medical device, wherein the second elongated body is separate from the first elongated body, wherein the second elongated body is configured to exert a proximally-directed force on a portion of the distal end of the implantable medical device, and wherein the second elongated body is disposed on the distal end of the implantable medical device away from an electrode disposed on the distal end of the implantable medical device, such that action of the second elongated body tends to urge the electrode towards contact with the tissue.

Example 76: the device of example 75, wherein the implantable medical device is configured to be implanted within a first chamber of a heart of the patient, wherein the first elongated body is configured to penetrate wall tissue of a second chamber of the heart, and wherein the action of the second elongated body tends to urge the electrode towards contact with the wall tissue of the first chamber.

Example 77: the device of example 76, wherein the distal end of the helix is configured to penetrate into a ventricular myocardium of the patient, and wherein the action of the second elongated body tends to urge the electrode towards contact with an atrial endocardium of the patient.

Example 78: the device of any of examples 75-76, wherein the second elongated body is configured as a partial helix.

Example 79: the device of example 78, wherein the partial helix is wound in a same direction as the helix of the first elongated body.

Example 80: the device of example 79, wherein the partial helix and the helix of the first elongated body are wound in a counter-clockwise direction.

Example 81: the device of any of examples 75-80, wherein the distal end of the implantable medical device defines a first recess configured to retain at least a portion of the second elongated body as the second elongated body exerts the proximally-directed force on the portion of the distal end of the implantable medical device.

Example 82: the device of example 81, wherein the distal end of the implantable medical device further comprises a therapeutic substance dispensing device disposed within the first recess.

Example 83: the device of example 82, wherein the therapeutic substance dispensing device comprises a monolithic controlled release device.

Example 84: the device of any of examples 82 and 83, wherein the implantable medical device further comprises a plurality of therapeutic substance dispensing devices disposed on the distal end of the implantable medical device.

Example 85: the device of any of examples 75-84, wherein the second elongated body is configured to cause the electrode to maintain contact against the tissue.

Example 86: the device of example 85, wherein the second elongated body comprises a Nickel-Titanium alloy.

Example 87: the device of any of examples 75-86, wherein the first elongated body resides in an inner space defined by the second elongated body and is substantially concentric with the second elongated body.

Example 88: the device of any of examples 75-87, wherein the second electrode is a button electrode.

Example 89: the device of any of examples 75-88, wherein the second elongated body is disposed on a peripheral region of the distal end of the implantable medical device.

Example 90: the device of any of examples 75-89, wherein the second electrode is disposed on a peripheral region of the distal end of the implantable medical device.

Example 91: the device of any of examples 75-90, wherein the first elongated body is disposed on the distal end of the implantable medical device between the second elongated body and the second electrode.

Example 92: the device of any of examples 75-91, wherein the second elongated body is configured to urge the second electrode towards the tissue of the patient.

Example 93: the device of example 92, wherein the second elongated body is configured to urge a first portion of the distal end of the implantable medical device away from the tissue of the patient while the second elongated body is urging the second electrode towards the tissue of the patient.

Various examples have been described. These and other examples are within the scope of the following claims.

1. A fixation device comprising:
a first elongated body extending distally from a distal end of an implantable medical device, the first elongated body comprising:
a helix having one or more coils, wherein a distal end of the helix is configured to penetrate into tissue of a patient; and
a second elongated body extending distally from the distal end of the implantable medical device, wherein the second elongated body is separate from the first elongated body, wherein the second elongated body is configured to exert a proximally-directed force on a portion of the distal end of the implantable medical device, and wherein the second elongated body is disposed on the distal end of the implantable medical device away from an electrode disposed on the distal end of the implantable medical device, such that action of the second elongated body tends to urge the electrode towards contact with the tissue.

2. The device of claim 1, wherein the implantable medical device is configured to be implanted within a first chamber of a heart of the patient, wherein the first elongated body is configured to penetrate wall tissue of a second chamber of the heart, and wherein the action of the second elongated body tends to urge the electrode towards contact with the wall tissue of the first chamber.

3. The device of claim 2, wherein the distal end of the helix is configured to penetrate into a ventricular myocardium of the patient, and wherein the action of the second elongated body tends to urge the electrode towards contact with an atrial endocardium of the patient.

4. The device of claim 1, wherein the second elongated body is configured as a partial helix.

5. The device of claim 4, wherein the partial helix is wound in a same direction as the helix of the first elongated body.

6. The device of claim 1, wherein the distal end of the implantable medical device defines a first recess configured to retain at least a portion of the second elongated body as the second elongated body exerts the proximally-directed force on the portion of the distal end of the implantable medical device.

7. (canceled)

8. The device of claim 1, wherein the second elongated body is configured to cause the electrode to maintain contact against the tissue.

9. The device of claim 1, wherein the first elongated body resides in an inner space defined by the second elongated body and is substantially concentric with the second elongated body.

10. (canceled)

11. The device of claim 1, wherein the second elongated body is disposed on a peripheral region of the distal end of the implantable medical device.

12. The device of claim 1, wherein the second electrode is disposed on a peripheral region of the distal end of the implantable medical device.

13. The device of claim 1, wherein the first elongated body is disposed on the distal end of the implantable medical device between the second elongated body and the second electrode.

14. The device of claim 1, wherein the second elongated body is configured to urge the second electrode towards the tissue of the patient.

15. The device of claim 14, wherein the second elongated body is configured to urge a first portion of the distal end of the implantable medical device away from the tissue of the patient while the second elongated body is urging the second electrode towards the tissue of the patient.

16. A device comprising:
an elongated housing that extends from a proximal end to a distal end, the elongated housing configured to be implanted wholly within a first chamber of a heart, the first chamber of the heart having wall tissue;
a first electrode extending distally from the distal end of the elongated housing, the first electrode comprising:
a first elongated body defining a helix, wherein the helix is configured to penetrate into wall tissue of a second chamber of the heart that is separate from the first chamber of the heart;
a second electrode disposed on the distal end of the elongated housing;
a second elongated body extending distally from the distal end of the elongated housing, wherein the second elongated body is connected to the distal end at a first location and when unstressed is furthest distally from the distal end of the elongated housing at a second location, wherein the second elongated body is separate from the first electrode and the second electrode, wherein the second elongated body is configured to maintain contact between the second electrode and the wall tissue of the first chamber without penetrate of the wall tissue of the first chamber by the second elongated body, and wherein the second elongated body is disposed on the distal end at a separation angle away from an electrode disposed on the distal end of the implantable medical device, wherein the separation angle comprises an angle between the electrode and the second location of the second elongated body; and
signal generation circuitry within the elongated housing, the signal generation circuitry being coupled to the first electrode and the second electrode, wherein the signal generation circuitry is configured to deliver cardiac pacing to the second chamber via the first electrode and the first chamber via the second electrode.

17. The device of claim 16, wherein the second elongated body is configured to resist rotation of the first electrode due to movement of the wall tissue of the first chamber.

18. The device of claim 16, wherein the first electrode is configured to penetrate into a ventricular myocardium of the patient, and wherein the second elongated body is configured to flexibly maintain contact between the second electrode and an atrial endocardium of the patient.

19. The device of claim 16, wherein the second elongated body is configured as a partial helix.

20. The device of claim 16, wherein the distal end of the elongated housing defines a first recess configured to retain at least a portion of the second elongated body as the second elongated body flexibly maintains contact between the second electrode and the wall tissue of the first chamber.

21. The device of claim 20, wherein the second elongated body further comprises a protrusion disposed at the second location of the second elongated body, wherein the distal end of the elongated housing further defines a second recess within the first recess, and wherein the second recess is configured to retain the protrusion of the second elongated body.

22. A method comprising:
delivering cardiac pacing from a device to a heart, wherein the device comprises:
an elongated housing extending from a proximal end to a distal end, the elongated housing configured to be implanted wholly within a first chamber of the heart, the first chamber of the heart having wall tissue;
a first electrode extending distally from the distal end of the elongated housing, the first electrode comprising:
a first elongated body defining a helix;
a second electrode disposed on the distal end of the elongated housing;
a second elongated body extending from the distal end of the elongated housing, wherein the second elongated body is connected to the distal end at a first location and when unstressed is furthest distally from the distal end of the elongated housing at a second location, wherein the second elongated body is separate from the first electrode and the second electrode, and wherein the second elongated body is configured to flexibly maintain contact between the second electrode and the wall tissue of the first chamber without penetration of the wall tissue of the first chamber by the second electrode or the second elongated body, wherein the second elongated body is disposed on the distal end at a separation angle away from an electrode disposed on the distal end of the implantable medical device, wherein the separation angle comprises an angle between the electrode and the second location of the second elongated body; and
signal generation circuitry within the elongated housing, the signal generation circuitry being coupled to the first electrode and the second electrode,
wherein delivering the cardiac pacing comprises:
delivering cardiac pacing to a second chamber via the first electrode; and
delivering cardiac pacing to the first chamber via the second electrode.
